(19) United States
(12) Patent Application Publication
(10) Pub. No.: US 2025/0367429 A1
(43) Pub. Date: Dec. 4, 2025

(54) THERMAL STIMULATION AND SUBSEQUENT COOLING FOR FULLY IMPLANTABLE LVAD CONTROLLER

(71) Applicant: BOSTON SCIENTIFIC SCIMED, INC., Maple Grove, MN (US)

(72) Inventors: Michael D. Eggen, Chisago City, MN (US); Yong K. Cho, Excelsior, MN (US); Avram Scheiner, Vadnais Heights, MN (US); Ramesh Raghupathy, New Brighton, MN (US); Thomas W. Radtke, Ramsey, MN (US)

(73) Assignee: BOSTON SCIENTIFIC SCIMED, INC., Maple Grove, MN (US)

(21) Appl. No.: 19/300,223

(22) Filed: Aug. 14, 2025

Related U.S. Application Data
(62) Division of application No. 16/935,433, filed on Jul. 22, 2020, now Pat. No. 12,409,313.

(51) Int. Cl. A61M 60/50; A61F 7/00; A61F 7/08; A61F 7/10; A61M 60/135

(52) U.S. Cl. CPC A61M 60/50; A61M 60/135; A61F 2007/0011; A61F 7/08; A61F 7/10; A61M 2205/18; A61M 2205/3372; A61M 2205/3584; A61M 2205/3606; A61M 2205/50; A61M 2205/8206; A61M 2230/50

(57) ABSTRACT

A method of cooling a mammal with an implantable blood pump. The method includes measuring a temperature of an internal controller, the internal controller being in communication with the implantable blood pump. An alert is generated if the temperature of the internal controller exceeds a predetermined temperature threshold. The internal controller and implantable blood pump may be implanted at different locations in the patient.

### CROSS-REFERENCE TO RELATED APPLICATION

This application is a divisional of U.S. patent application Ser. No. 16/935,433, filed Jul. 22, 2020, the disclosure of which is incorporated herein by reference.

### FIELD

The present technology is generally related to a method and system of thermally stimulating a patient with an implantable controller for an implantable blood pump.

### BACKGROUND

With the advent of fully implantable blood pump systems comes a controller that is implanted within the body of patient. The controller has an internal battery that provides power to the implantable blood pump. Like any controller with a battery, the controller gives off heat as it provides power to the pump. As the power increases, for example, in the presence of thrombus, more heat is transferred from the controller to the patient, which may cause discomfort and even pain.

### SUMMARY

The techniques of this disclosure generally relate to a method and system of thermally stimulating a patient with an implantable controller for an implantable blood pump.

In one aspect, the present disclosure provides a method of cooling a patient with an implantable blood pump. The method includes measuring a temperature of an internal controller, the internal controller being in communication with the implantable blood pump. An alert is generated if the temperature of the internal controller exceeds a predetermined temperature threshold.

In another aspect of this embodiment, the alert instructs the patient to place a heating pad on a body of the patient.

In another aspect of this embodiment, the alert instructs the patient to place the heating pad on the back of a neck of the body of the patient.

In another aspect of this embodiment, wherein the alert instructs the patient to go to a hospital.

In another aspect of this embodiment, the predetermined temperature threshold is 39 degrees Celsius.

In another aspect of this embodiment, the predetermined temperature threshold is between 39 degrees Celsius and 43 degrees Celsius.

In another aspect of this embodiment, the predetermined temperature threshold is 43 degrees Celsius.

In another aspect of this embodiment, the method further includes communicating the measured temperature to an external controller in communication with the internal controller.

In another aspect of this embodiment, the alert is generated by the external controller.

In another aspect of this embodiment, the alert instructs the patient to thermally stimulate the back of a neck of the body of the patient.

In one aspect, a system for cooling a patient with an implantable blood pump includes an internal controller configured to be in communication with the implantable blood pump. An external controller is configured to be in communication with the internal controller, the external controller being configured to generate an alert if a temperature of the internal controller exceeds a predetermined temperature threshold.

In another aspect of this embodiment, the internal controller includes at least one temperature sensor configured to measure a temperature of the internal controller.

In another aspect of this embodiment, the internal controller is configured to communicate the measured temperature to the external controller.

In another aspect of this embodiment, the alert instructs the patient to place the heating pad on the back of a neck of the body of the patient.

In another aspect of this embodiment, the alert instructs the patient to go to a hospital.

In another aspect of this embodiment, the predetermined temperature threshold is 39 degrees Celsius.

In another aspect of this embodiment, the predetermined temperature threshold is between 39 degrees Celsius and 43 degrees Celsius.

In another aspect of this embodiment, the predetermined temperature threshold is 43 degrees Celsius.

In another aspect of this embodiment, the alert instructs the patient to thermally stimulate the back of a neck of the body of the patient.

In one aspect, a method of cooling a patient with an implantable blood pump includes measuring a temperature of an internal controller with a temperature sensor coupled to the internal controller, the internal controller being in communication with the implantable blood pump. The measured temperature is communicated to an external controller in communication with the internal controller. An alert is generated if the temperature of the internal controller exceeds a temperature greater than 39 degrees Celsius, the alert notifying the patient to thermally stimulate a back of the neck of the patient.

### BRIEF DESCRIPTION OF THE DRAWINGS

A more complete understanding of the present invention, and the attendant advantages and features thereof, will be more readily understood by reference to the following detailed description when considered in conjunction with the accompanying drawings wherein:

FIG. 1 is an internal system view of an implantable blood pump with a TETS receiver source constructed in accordance with the principles of the present application;

FIG. 2 is an external view of a TETS transmitter and a controller of the system shown in FIG. 1; and

FIG. 3 is a flow chart showing the steps of an exemplary method of the present application.

### DETAILED DESCRIPTION

It should be understood that various aspects disclosed herein may be combined in different combinations than the combinations specifically presented in the description and accompanying drawings. It should also be understood that, depending on the example, certain acts or events of any of the processes or methods described herein may be performed in a different sequence, may be added, merged, or left out altogether (e.g., all described acts or events may not be necessary to carry out the techniques). In addition, while certain aspects of this disclosure are described as being performed by a single module or unit for purposes of clarity, it should be understood that the techniques of this disclosure may be performed by a combination of units or modules associated with, for example, a medical device.

In one or more examples, the described techniques may be implemented in hardware, software, firmware, or any combination thereof. If implemented in software, the functions may be stored as one or more instructions or code on a computer-readable medium and executed by a hardware-based processing unit. Computer-readable media may include non-transitory computer-readable media, which corresponds to a tangible medium such as data storage media (e.g., RAM, ROM, EEPROM, flash memory, or any other medium that can be used to store desired program code in the form of instructions or data structures and that can be accessed by a computer).

Instructions may be executed by one or more processors, such as one or more digital signal processors (DSPs), general purpose microprocessors, application specific integrated circuits (ASICs), field programmable logic arrays (FPGAs), or other equivalent integrated or discrete logic circuitry. Accordingly, the term “processor” as used herein may refer to any of the foregoing structure or any other physical structure suitable for implementation of the described techniques. Also, the techniques could be fully implemented in one or more circuits or logic elements.

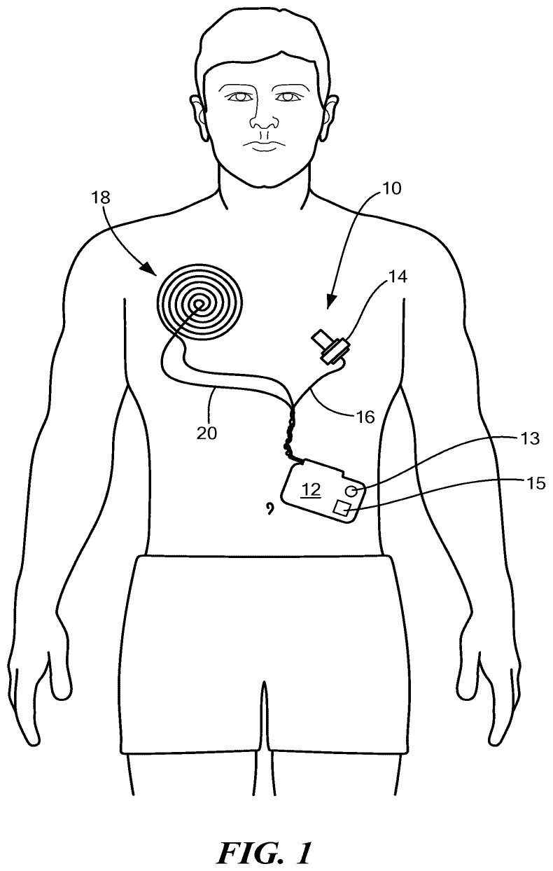

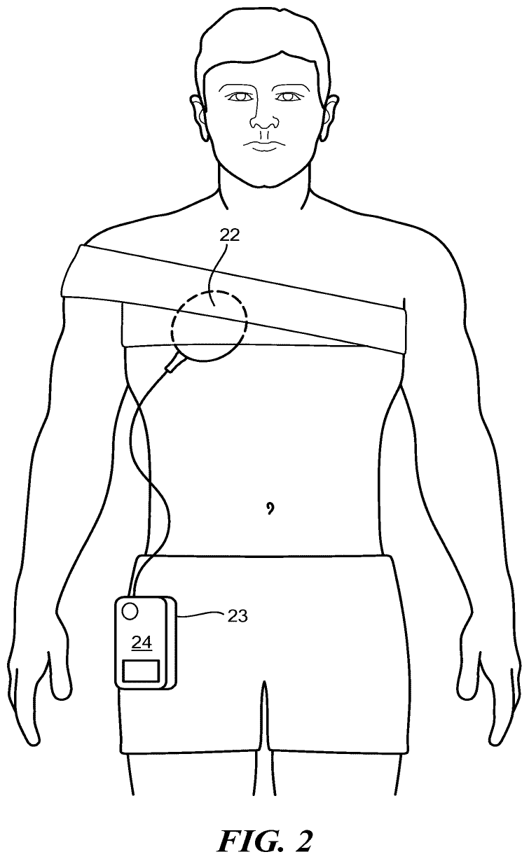

Referring now to the drawings in which like reference designators refer to like elements there is shown in FIGS. 1 and 2 an exemplary mechanical circulatory support device (MCSD) constructed in accordance with the principles of the present application and designated generally as “10.” The MCSD 10 may be fully implantable within a patient, whether human or animal, which is to say there are no percutaneous connections between the implanted components of the MCSD 10 and the components outside of the body of the patient. In the configuration shown in FIG. 1, the MCSD 10 includes an internal controller 12 implanted within the body of the patient. The internal controller 12 includes a control circuit having processing circuitry configured to control operation of an implantable blood pump 14. The internal controller 12 may include an internal power source 13, configured to power the components of the controller and provide power to one or more implantable medical devices, for example, the implantable blood pump, such as a ventricular assist device (“VAD”) 14 implanted within the left ventricle of the patient's heart. The power source 13 may include a variety of different types of power sources including an implantable battery. The internal controller 12 may also include one or more temperature sensors 15 configured to measure a temperature of the controller 12 during operation of VAD 14. VADs 14 may include centrifugal pumps, axial pumps, or other kinds electromagnetic pumps configured to pump blood from the heart to blood vessels to circulate around the body. One such centrifugal pump is the HVAD and is shown and described in U.S. Pat. No. 7,997,854, the entirety of which is incorporated by reference. One such axial pump is the MVAD and is shown and described in U.S. Pat. No. 8,419,609, the entirety of which is incorporated herein by reference. In an exemplary configuration, the VAD 14 is electrically coupled to the internal controller 12 by one or more implanted conductors 16 configured to provide power to the VAD 14, relay one or more measured feedback signals from the VAD 14, and/or provide operating instructions to the VAD 14.

Continuing to refer to FIG. 1, a receiving or internal coil 18 may also be coupled to the internal controller 12 by, for example, one or more implanted conductors 20. In an exemplary configuration, the receiving coil 18 may be implanted subcutaneously proximate the thoracic cavity, although any subcutaneous position may be utilized for implanting the receiving coil 18. The receiving coil 18 is configured to be inductively powered through the patient's skin by a transmission or external coil 22 (seen in FIG. 2) disposed opposite the receiving coil 18 on the outside/exterior of the patient's body. For example, as shown in FIG. 2, a transmission coil 22 may be coupled to an external controller 23 having a power source 24, for example, a portable battery carried by the patient or wall power. In one configuration, the battery is configured to generate a radiofrequency signal for transmission of energy from the transmission coil 22 to the receiving coil 18. The receiving coil 18 may be configured for transcutaneous inductive communication with the transmission coil 22.

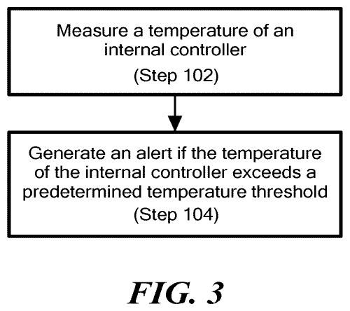

Referring now to FIG. 3, in which a method of cooling a patient with pump 14 and controller 12 is shown. The method includes measuring a temperature of the internal controller 12 (Step 102). For example, the controller 12 may include the one or more temperature sensors 15 configured to measure a temperature of the controller 12. As more power to the pump 14 is required, the temperature of the controller 12 may increase beyond a predetermined temperature threshold. If the temperature of the internal controller 12 exceeds the predetermined temperature threshold an alert may generated that instructs the patient, whether human or animal, with the implanted pump 14 to thermally stimulate a portion of the patient's body (Step 104). For example, the alert may instruct the patient to apply a heating pad to the back of the neck, which has the effect of cooling the body. In particular, a heating pad or thermal stimulus at a temperature of 42-44 degrees Celsius placed on the back of the neck may reduce the patient's core temperature by approximately 2 degrees Celsius. This may reduce the temperature of the controller 12 by 33% if heated 6 degrees Celsius for example, and allows time for the patient to get to the hospital, while minimizing thermal tissue damage and pain. In other configurations, the alert may instruct the user to bring cold fluids, to place a cold vest on the body, or other methods to cool the body. In an exemplary configuration, the internal controller 12 communicates with the external controller 23 which generates the alert. For example, the internal controller 12 may commutate via wireless communication, for example, Bluetooth, with the external controller 23 concerning the measured temperature. The alert may be an audio or text alert that notifies the patient of either the overheated controller 12 or with a specific instruction to cool the patient's body and/or to go to a hospital or see a clinician. Exemplary temperature thresholds may be 2 degrees Celsius above body temperature, between 2 and 7 degrees Celsius above body temperature, or 7 degrees Celsius above body temperature.

It will be appreciated by persons skilled in the art that the present invention is not limited to what has been particularly shown and described herein above. In addition, unless mention was made above to the contrary, it should be noted that all of the accompanying drawings are not to scale. A variety of modifications and variations are possible in light of the above teachings without departing from the scope and spirit of the invention, which is limited only by the following claims.

What is claimed is:

1. A method of managing temperature for a patient with an implantable blood pump, comprising:
measuring a temperature of an internal controller, the internal controller being in communication with the implantable blood pump;
generating an alert if the temperature of the internal controller exceeds a predetermined temperature threshold.

2. The method of claim 1, wherein the alert instructs the patient to place a heating pad on a body of the patient.

3. The method of claim 2, wherein the alert instructs the patient to place the heating pad on the back of a neck of the body of the patient.

4. The method of claim 1, wherein the alert instructs the patient to go to a hospital.

5. The method of claim 1, wherein the predetermined temperature threshold is 39 degrees Celsius.

6. The method of claim 1, wherein the predetermined temperature threshold is between 39 degrees Celsius and 43 degrees Celsius.

7. The method of claim 1, wherein the predetermined temperature threshold is 43 degrees Celsius.

8. The method of claim 1, wherein the method further includes the internal controller communicating concerning the measured temperature to an external controller in communication with the internal controller.

9. The method of claim 8, wherein the alert is generated by the external controller.

10. The method of claim 1, wherein the alert instructs thermally stimulating the back of a neck of the body of the patient.

11. The method of claim 1, wherein the internal controller is physically separate from the implantable pump.

12. A method of managing temperature a patient with an implantable blood pump, comprising:
a) measuring a temperature of an internal controller with a temperature sensor coupled to the internal controller, the internal controller being in communication with and physically separate from the implantable blood pump;
b) the internal controller communicating concerning the measured temperature to an external controller in communication with the internal controller;
c) responsive to b), the external controller generating an alert notifying the patient to thermally stimulate a body part of the patient.

13. The method of claim 12, wherein the alert notifies the patient to thermally stimulate a neck of the patient.

14. The method of claim 13, wherein the alert notifies the patient to apply a heating pad to the neck of the patient.

15. The method of claim 12, wherein the alert notifies the patient to apply a cooling vest.

16. The method of claim 12, wherein step b) includes communicating the measured temperature of the internal controller.

17. The method of claim 12, wherein the internal controller is coupled to an implantable coil adapted to receive transcutaneous power, and the internal controller comprises a battery for powering the implantable pump.
